(19) United States
(12) Patent Application Publication
(10) Pub. No.: US 2023/0315933 A1
(43) Pub. Date: Oct. 5, 2023

(54) SWIVELING TABLE SYSTEM AND METHOD THEREOF

(71) Applicant: TG3D Studio, Taipei City (TW)

(72) Inventors: Hsien CHOU, Taipei City (TW); Cheng-Kai LIN, Taipei City (TW)

(21) Appl. No.: 18/190,997

(22) Filed: Mar. 28, 2023

(30) Foreign Application Priority Data
Apr. 1, 2022 (TW) 111112800

(51) Int. Cl. G06F 30/17; G01N 33/36

(52) U.S. Cl. CPC G06F 30/17; G01N 33/367

(57) ABSTRACT

The swiveling table system comprises a 3D sensor and a swiveling table device disposed on a base having a central turntable and a lifting platform. The central turntable includes a central tray and a central rotation column coupled with the central tray for rotating the central tray. The lifting platform includes an outer ring tray and a lifting column coupled with the outer ring tray for lifting the outer ring tray. The outer ring tray and the central tray are coplanar. When a fabric is placed on the coplanar and the lifting column is controlled to lower the outer ring tray in a free-fall manner, the central rotating column rotates the central tray and the 3D sensor captures a rotating image of the fabric to build a 3D model of the fabric.

### CROSS-REFERENCE TO RELATED APPLICATION

This application claims priority to Taiwan Application Serial Number 111112800, filed Apr. 1, 2022, which is herein incorporated by reference.

### BACKGROUND OF THE INVENTION

### Field of Invention

The present invention relates to a swiveling table system, in particular to a swiveling table system for building a 3D model of fabric to assist in calculating the drape coefficient.

### Description of Related Art

Traditionally, there is no doubt about the upper body effect of fabrics with good drape. Today, the drape coefficient is often used to evaluate the possible drape.

However, the design and development of fabric materials have progressed rapidly, and fabrics have changed from less diverse forms in the past to diversified products designed to meet consumer needs. And it is more multifunctional, and functional fabrics, such as antibacterial and deodorant, sweat-wicking, breathable and waterproof, anti-ultraviolet, etc., or include certain mechanical properties, such as strength, elongation, modulus, heat shrinkage and water permeability coefficients and so on.

Therefore, how to check and judge which fabric has better drape at any time in the process of technology development is an urgent issue in the industry.

### SUMMARY

According to an aspect of the present invention, a swiveling table system comprises a 3D sensor and a swiveling table device. The swiveling table device further comprises a central turntable disposed on a base and a lifting platform. The central turntable includes a central rotation column and a central tray. The central rotation column is coupled to the central tray for rotating the central tray. The lifting platform disposed on the base. The lifting platform includes a lifting column and an outer ring tray. The lifting column is coupled to the outer ring tray for lifting the outer ring tray. When the outer ring tray and the central tray are coplanar, the outer ring tray surrounds the central tray and is not coupled with the central tray. When a fabric is placed on the coplanar and the lifting column is controlled to lower the outer ring tray in a free-fall manner to make the outer ring tray drop from the position coplanar with the central tray to a descending position in a direction perpendicular to the base, the central rotating column rotates the central tray and the 3D sensor captures a rotating image of the fabric to establish a 3D model of the fabric.

In an embodiment, the central tray further includes a fabric plate and a pressure plate, wherein the fabric plate is configured to place the fabric, and the pressure plate is configured to fix the fabric.

In an embodiment, warp scales are formed on the fabric plate configured to position the warp and weft directions of the fabric.

In an embodiment, the pressure plate includes a magnetic element is configured to fix the fabric on the fabric plate by magnetic attraction.

In an embodiment, the lifting column further includes a fixing base disposed on the base, a sliding rail disposed on the fixing base, and a lifting rod coupled to the outer ring tray, wherein the lifting rod includes a sliding mechanism connected to the sliding rail, and the sliding mechanism moves on the sliding rail to lift the lifting rod.

In an embodiment, both the lifting rod and the fixing base have a pin hole respectively.

In an embodiment, when the pin hole on the lifting rod is aligned with the pin hole on the fixing base, a latch is configured to insert into the pin hole on the lifting rod and the pin hole on the fixing base to fix the lifting rod on the fixing base, wherein when the lifting rod is fixed on the fixing base, the outer ring tray and the central tray are coplanar.

In an embodiment, when the latch is removed from the pin hole on the lifting rod and the pin hole on the fixing base, the lifting rod slides downwards along the sliding rail in a free-fall manner to make the outer ring tray drop from the position coplanar with the central tray to the descending position.

In an embodiment, a buffer disposed on the base is configured to buffer the lifting rod falling in the free-fall manner.

In an embodiment, the swiveling table system of claim 1 further comprises a first 2D sensor configured to capture a 2D image of a front view of the fabric when the fabric is rotating.

In an embodiment, the swiveling table system of claim 1 further comprises a second 2D sensor configured to capture a 2D image of a top view of the fabric when the fabric is placed on the coplanar.

In an embodiment, the swiveling table system of claim 1 further comprises a backplane vertically disposed on the base, wherein the backplane and the base form an “L” shape appearance.

In an embodiment, the backplane and the base are configure to be a green screen.

A method for operating the swiveling table system to build a 3D model of a fabric comprises to make the outer ring tray coplanar with the central tray, to place the fabric on the coplanar, to control the lifting column to lower the outer ring tray in a free-fall manner to make the outer ring tray drop from the position coplanar with the central tray to the descending position in the direction perpendicular to the base, to activate the central rotating column to rotate the central tray, to activate the 3D sensor to capture the rotating image of the fabric, and to build the 3D model of the fabric according to the rotating image of the fabric.

Accordingly, the swiveling table device includes a lifting platform and a central turntable. The lifting platform slides down in a free-fall manner to make the fabrics placed on the outer ring tray to hang down in a free-fall manner. At the same time, the central turntable rotates the fabric on the central tray, and captures the rotating image of the fabric to reconstruct the 3D model of the fabric, so as to calculate the drape coefficient of the fabric. Since the fabric is draped in a natural way to create the 3D model, the 3D model of fabric is very closer to the actual use situation. The user may actually feel the drape situation of the fabric through the 3D model of the fabric. Therefore, the 3D model of the fabric may provide more accurate reference data for developing fabric.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

In order to make the above and other objects, features and advantages of the disclosure more comprehensible, several embodiments accompanied with figures are described in detail below.

FIG. 1 is a schematic diagram of a swiveling table system according to an embodiment.

FIG. 2 is a schematic diagram of a swiveling table device according to an embodiment.

FIG. 3 is a schematic diagram when the lifting column of the lifting platform lowers the outer ring tray.

FIG. 4A is a schematic diagram of a central turntable according to an embodiment.

FIG. 4B is an exploded schematic diagram of a central turntable according to an embodiment.

FIG. 5A is a schematic diagram of a lifting platform according to an embodiment.

FIG. 5B is an exploded schematic diagram of a lifting column according to an embodiment.

FIG. 6 is a flow chart for building a 3D model of fabric using a swiveling table system to rotate the fabric according to an embodiment.

### DETAILED DESCRIPTION

Reference will now be made in detail to the present preferred embodiments of the disclosure, examples of which are illustrated in the accompanying drawings. Wherever possible, the same reference numbers are used in the drawings and the description to refer to the same or like parts.

To comprehensively describe the disclosure in detail, reference may be made to the accompanying drawings and various embodiments. Meanwhile, components and steps known by the public are not described in the embodiments to prevent unnecessary limitations from being imposed to the disclosure.

Terms such as “couple” or “connect” used in the embodiments may refer to two or more components being in physical or electrical contact with each other “directly”, two or more components being in physical or electrical contact with each other “indirectly”, or acting of two or more components with each other.

The foregoing and other technical content, features, and effects of the present invention can be clearly presented below in detailed descriptions of embodiments with reference to the drawings.

The present application provides a swiveling table system, which can be used to rotate the fabric in the process of fabric development to reconstruct the 3D model of the fabric and calculate the drape coefficient of the fabric based on the 3D model. The lifting mechanism of the swiveling table system may realize the natural drape of the fabric. Accordingly, in addition to allowing the user to feel the drape of the fabric, the present application may also make the established 3D model of the fabric closer to the actual situation and provide users with more accurate reference data for developing fabric.

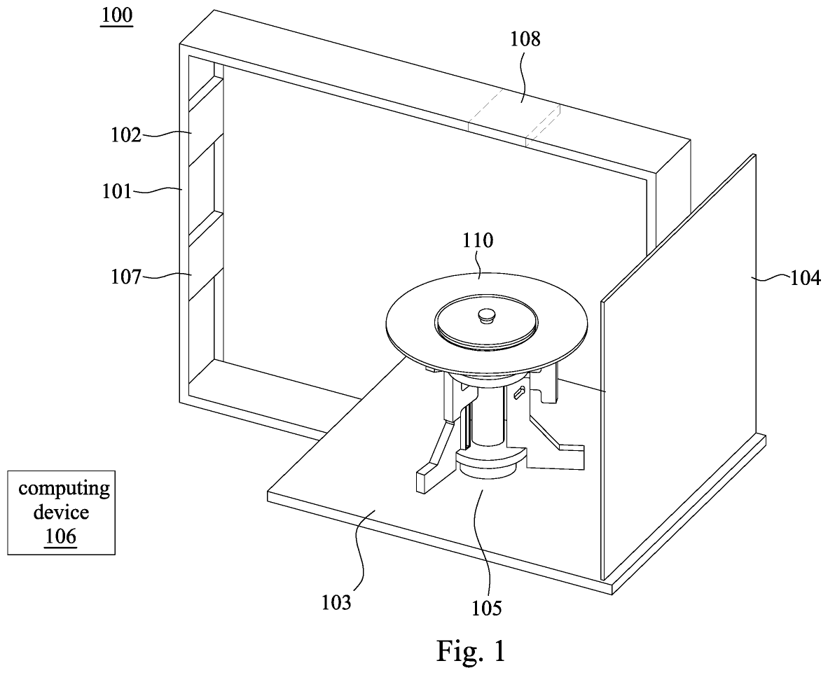

FIG. 1 is a schematic diagram of a swiveling table system according to an embodiment, which is used to establish a 3D model of fabric to assist in calculating the drape coefficient. The swiveling table system 100 includes a fixed shelf 101, a 3D sensor 102, a base 103, a backplane 104 and a swiveling table device 105. The fixed shelf 101 is used to support the base 103 and the backplane 104, so that the backplane 104 is vertically arranged on the base 103 to form an “L” shaped appearance with the base 103. The swiveling table device 105 is fixed on the base 103, and a fabric can be placed on the tray 110 of the swiveling table device 105. The swiveling table device 105 rotates the fabric, and the 3D sensor 102 captures the rotating image of the fabric to reconstruct a 3D model of the fabric. This 3D model is used to calculate the fabric drape coefficient. The 3D sensor 102 is arranged on the fixed shelf 101 corresponding to the position of the swiveling table device 105 to capture the rotating image of the fabric. In one embodiment, the 3D sensor 102 is connected to a computing device 106 in a wireless or wired connection. The computing device 106 reconstructs the 3D model of the fabric according to the rotation image of the fabric captured by the 3D sensor 102, and calculates the fabric drape coefficient according to the reconstructed 3D model.

In one embodiment, a first 2D sensor 107 and a second 2D sensor 108 are further provided on the fixed shelf 101 for capturing 2D images of the fabric. In one embodiment, the first 2D sensor 107 is disposed in front of the swiveling table device 105 to capture a 2D image of a front view of the fabric when it is draped. The second 2D sensor 108 is disposed above the swiveling table device 105 to capture a 2D image of a top view of the fabric when it is placed on the tray 110. In one embodiment, the base 103 and the backplane 104 can be set as a green background to form a green screen for the 3D sensor 102, the first 2D sensor 107 and the second 2D sensor 108 to capture images. The green background may be replaced to remove unnecessary background images or add extra background images.

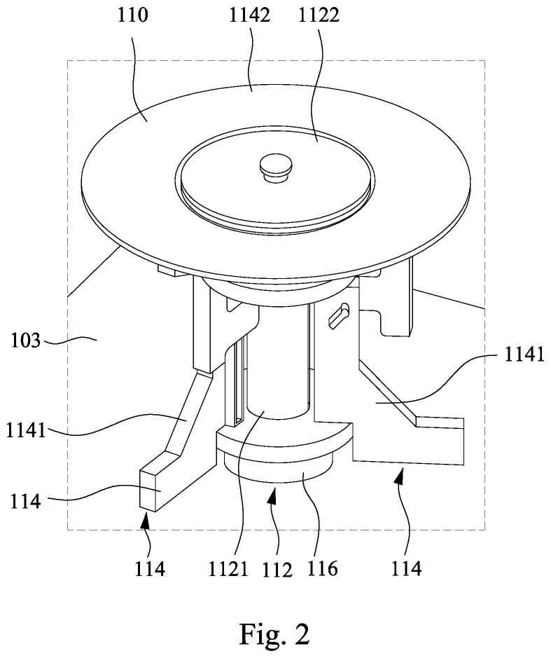

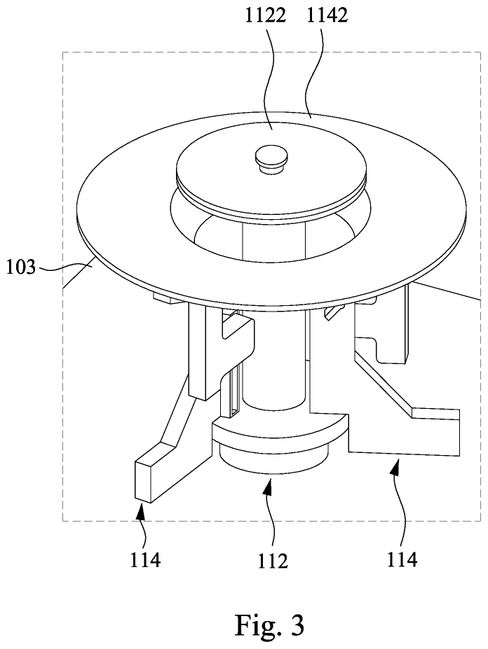

FIG. 2 is a schematic diagram of a swiveling table device according to an embodiment. The swiveling table device 105 includes a central turntable 112 and a lifting platform 114. In one embodiment, the central turntable 112 includes a central rotating column 1121 and a central tray 1122. The central rotating column 1121 is coupled to the central tray 1122 to rotate the central tray 1122 independently. In one embodiment, the lifting platform 114 includes a lifting column 1141 and an outer ring tray 1142. The lifting column 1141 is coupled to the outer ring tray 1142 to lift the outer ring tray 1142 independently. In one embodiment, when the central tray 1122 and the outer ring tray 1142 are coplanar, the tray 110 for placing fabric is formed. The outer ring tray 1142 surrounds the central tray 1122 and is not coupled with the central tray 1122. FIG. 3 is a schematic diagram of the central tray 1122 protruding from the outer ring tray 1142 when the lifting column 1141 of the lifting platform 114 lowers the outer ring tray 1142. Accordingly, when fabric is placed on the tray 110 and the lifting column 1141 is controlled to lower the outer ring tray 1142 in a free-fall manner, the outer ring tray 1142 will drop from the position coplanar with the central tray 1122 to a descending position in a direction perpendicular to the base 103 to realize the fabric naturally draped.

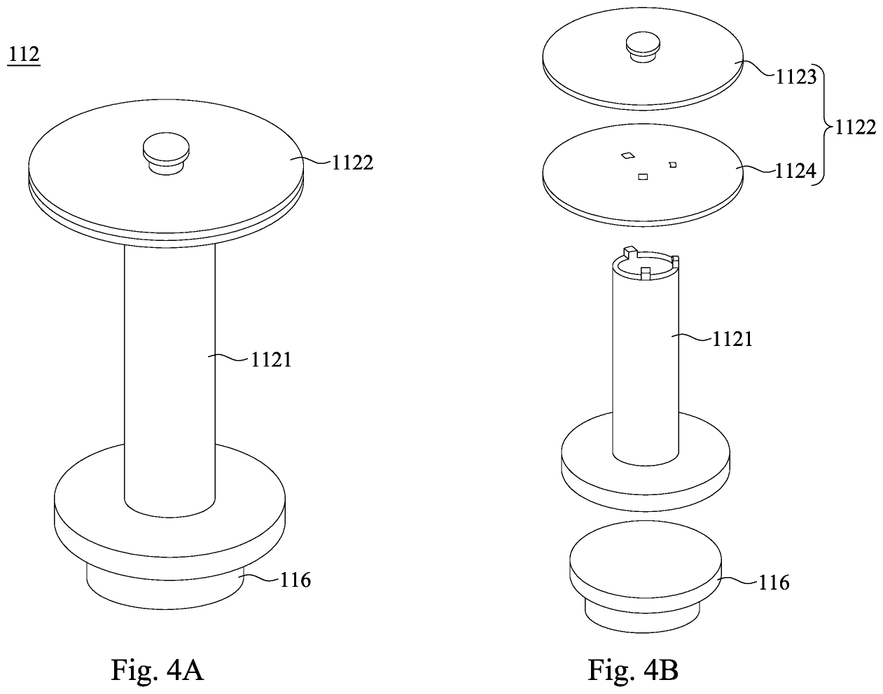

FIG. 4A is a schematic diagram of a central turntable according to an embodiment. FIG. 4B is an exploded schematic diagram of a central turntable according to an embodiment. Please refer to FIG. 4A and FIG. 4B. The central turntable 112 is disposed on the base 103. In one embodiment, the central turntable 112 includes a central rotating column 1121 and a central tray 1122. The central rotating column 1121 is rotated by a rotating element 116 disposed below the central rotating column 1121 to drive the central tray 1122 to rotate, so as to provide the 3D sensor 102 to capture the rotating image of the fabric. In one embodiment, the rotating element 116 is a rotating motor whose rotational speed can be controlled by an external computer. In one embodiment, the central tray 1122 further includes a fabric plate 1124 and a pressure plate 1123. The fabric is placed on the fabric plate 1124. In one embodiment, warp scales are engraved on the fabric plate 1124 for positioning the warp and weft directions of the fabric. Pressure plate 1123 is used for fixing the fabric placed on the fabric plate 1124. In one embodiment, the pressure plate 1123 may include a magnetic element to quickly fix the fabric on the fabric plate 1124 by means of magnetic attraction. In one embodiment, when the fabric is placed on the fabric plate 1124, the fabric is aligned according to the warp scales engraved on the fabric plate 1124 to position the warp and weft directions of the fabric. Then, the pressure plate 1123 is fixed on the fabric plate 1124 in a magnetically attractive manner to fix the fabric on the fabric plate 1124.

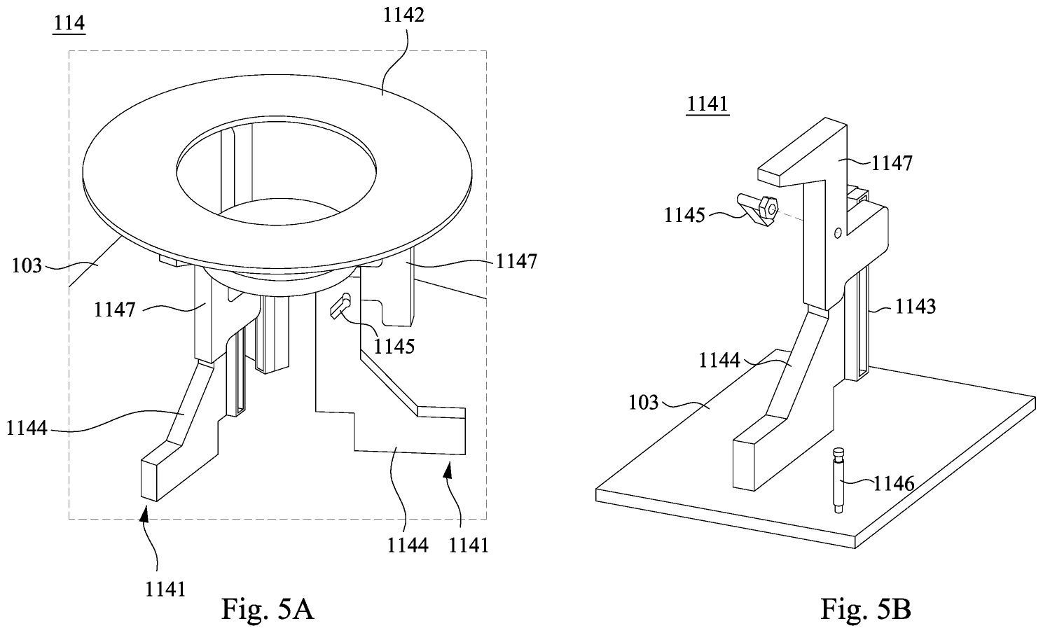

FIG. 5A is a schematic diagram of a lifting platform according to an embodiment. FIG. 5B is an exploded schematic diagram of a lifting column according to an embodiment. Please refer to FIG. 5A and FIG. 5B together. The lifting platform 114 is disposed on the base 103. In one embodiment, the lifting platform 114 includes three lifting columns 1141 and an outer ring tray 1142. In one embodiment, the three lifting columns 1141 support the outer ring tray 1142 together to stabilize the outer ring tray 1142. In one embodiment, the lifting column 1141 further includes a lifting rod 1147, a sliding rail 1143 and a fixing base 1144. The lifting rod 1147 is coupled to the outer ring tray 1142. The fixing base 1144 is fixed on the base 103. The sliding rail 1143 is disposed on the fixing base 1144. The lifting rod 1147 includes a sliding mechanism connected to the sliding rail 1143. The sliding mechanism can move on the sliding rail 1143 to raise or lower the lifting rod 1147. When the lifting rod 1147 rises, the lifting rod 1147 drives the outer ring tray 1142 to rise. When the lifting rod 1147 descends, the lifting rod 1147 drives the outer ring tray 1142 to descend.

In one embodiment, the lifting platform 114 further comprises a latch 1145 and each the lifting rod 1147 and the fixing base 1144 has a pin hole. When the pin hole disposed on the lifting rod 1147 is in alignment with the pin hole disposed on the fixing base 1144, the latch 1145 is penetrated into the pin holes disposed on the lifting rod 1147 and the fixing base 1144 simultaneously to make the lifting rod 1147 be fixed on the fixing base 1144. In one embodiment, the position where the lifting rod 1147 is fixed on the fixing base 1144 is, but not necessarily, the lifting column 1141 slides to the top of the sliding rail 1143. In one embodiment, when the lifting rod 1147 is fixed on the fixing base 1144, the outer ring tray 1142 is coplanar with the central tray 1122 to form the tray 110. When the latch 1145 is moved out from the pin holes on the lifting rod 1147 and the fixing base 1144, the lifting rod 1147 slides down along the sliding rail 1143 on the fixing base 1144 in a free-fall manner to drive the outer ring tray 1142 to slide down simultaneously. Accordingly, the outer ring tray 1142 drops from the coplanar position with the central tray 1122 to a lowering position in a direction perpendicular to the base 103. At this time, because the outer ring tray 1142 slides downward, the part of the fabric placed on the outer ring tray 1142 may hang down naturally in the way of free fall. In another embodiment, the base 103 further comprises a buffer 1146. The buffer 1146 is disposed on the base 103 to provide buffer for the lifting rod 1147. The buffer 1146 may prevent the lifting rod 1147 from directly hitting the base 103 to damage the outer ring tray 1142 when the lifting rod 1147 slides down along the sliding rail 1143 on the fixing base 1144 in a free-fall manner.

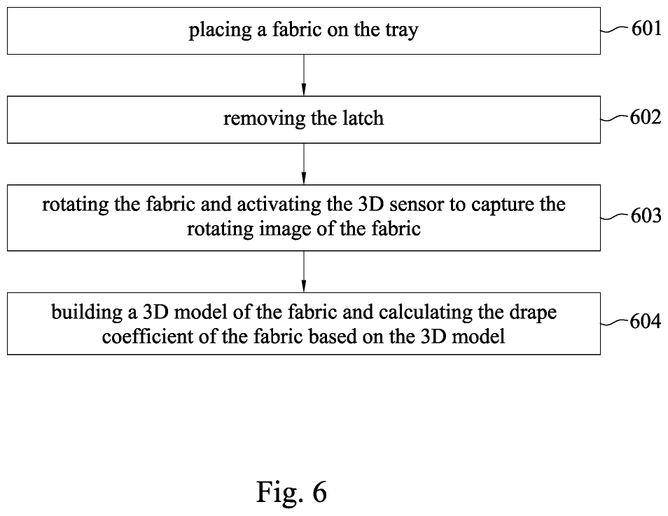

FIG. 6 is a flow chart for reconstructing a 3D model of fabric using a swiveling table system to rotate the fabric according to an embodiment. Please refer to FIG. 1 to FIG. 6 together. In step 601, a fabric is placed on the tray 110. In one embodiment, before reconstructing the 3D model of the fabric, the outer ring tray 1142 of the lifting platform 114 is aligned to the central tray 1122 of the central turntable 112. That is, the outer ring tray 1142 and the central tray 1122 will form a plane, the tray 110. When the fabric is placed on the tray 110, the fabric is aligned according to the warp scales engraved on the fabric plate 1124 to position the warp and weft directions of the fabric. Then, the pressure plate 1123 is fixed on the fabric plate 1124 in a magnetically attractive manner to fix the fabric on the fabric plate 1124. Then, in step 602, the latch is removed. In one embodiment, when the latch 1145 is moved out from the pin holes on the lifting rod 1147 and the fixing base 1144, the lifting rod 1147 slides down along the sliding rail 1143 on the fixing base 1144 in a free-fall manner to drive the outer ring tray 1142 to slide down simultaneously. At this time, because the outer ring tray 1142 slides downward, the part of the fabric placed on the outer ring tray 1142 hangs down naturally in the way of free fall. In step 603, the fabric is rotated and the 3D sensor is activated to capture the rotating image of the fabric. In one embodiment, the rotating element 116 of the central turntable 112 is controlled to rotate the central rotating column 1121 to drive the central tray 1122 to rotate, thereby driving the fabric to rotate and providing the 3D sensor 102 to capture the rotating image of the fabric. In step 604, a 3D model of the fabric is established and drape coefficient is calculated based on the 3D model. In one embodiment, the rotating image of the fabric captured by the 3D sensor 102 is sent to a back end to establish the 3D model of the fabric and to calculate the drape coefficient based on the 3D model.

Accordingly, the swiveling table device includes a lifting platform and a central turntable. The lifting platform slides down in a free-fall manner to make the fabrics placed on the outer ring tray to hang down in a free-fall manner. At the same time, the central turntable rotates the fabric on the central tray, and captures the rotating image of the fabric to reconstruct the 3D model of the fabric, so as to calculate the drape coefficient of the fabric. Since the fabric is draped in a natural way to create the 3D model, the 3D model of fabric is very closer to the actual use situation. The user may actually feel the drape situation of the fabric through the 3D model of the fabric. Therefore, the 3D model of the fabric may provide more accurate reference data for developing fabric.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims and their equivalents.

What is claimed is:

1. A swiveling table system, comprising:
a 3D sensor; and
a swiveling table device disposed on a base, wherein the swiveling table device comprises:
a central turntable disposed on the base, wherein the central turntable includes a central rotating column and a central tray, and the central rotation column is coupled to the central tray for rotating the central tray; and
a lifting platform disposed on the base, wherein the lifting platform includes a lifting column and an outer ring tray, and the lifting column is coupled to the outer ring tray for lifting the outer ring tray,
wherein when the outer ring tray and the central tray are coplanar, the outer ring tray surrounds the central tray and is not coupled with the central tray, and
when a fabric is placed on the coplanar and the lifting column is controlled to lower the outer ring tray in a free-fall manner to make the outer ring tray drop from a position coplanar with the central tray to a descending position in a direction perpendicular to the base, the central rotating column rotates the central tray and the 3D sensor captures a rotating image of the fabric, wherein the rotating image of the fabric is configured to build a 3D model of the fabric.

2. The swiveling table system of claim 1, wherein the central tray further includes a fabric plate and a pressure plate, wherein the fabric plate is configured to place the fabric, and the pressure plate is configured to fix the fabric.

3. The swiveling table system of claim 2, wherein warp scales are formed on the fabric plate configured to position the warp and weft directions of the fabric.

4. The swiveling table system of claim 2, wherein the pressure plate includes a magnetic element configured to fix the fabric on the fabric plate by magnetic attraction.

5. The swiveling table system of claim 1, wherein the lifting column comprises:
a fixing base disposed on the base;
a sliding rail disposed on the fixing base; and
a lifting rod coupled to the outer ring tray, wherein the lifting rod includes a sliding mechanism connected to the sliding rail, and the sliding mechanism moves on the sliding rail to lift the lifting rod.

6. The swiveling table system of claim 5, wherein each of the lifting rod and the fixing base has a pin hole.

7. The swiveling table system of claim 6, wherein the lifting platform further comprises a latch, and
wherein when the pin hole on the lifting rod is aligned with the pin hole on the fixing base, the latch is configured to insert into the pin hole on the lifting rod and the pin hole on the fixing base to fix the lifting rod on the fixing base, and
wherein when the lifting rod is fixed on the fixing base, the outer ring tray and the central tray are coplanar.

8. The swiveling table system of claim 7, wherein when the latch is removed from the pin hole on the lifting rod and the pin hole on the fixing base, the lifting rod slides downwards along the sliding rail in a free-fall manner to make the outer ring tray drop from the position coplanar with the central tray to the descending position.

9. The swiveling table system of claim 8, wherein the base further includes a buffer configured to buffer the lifting rod falling in the free-fall manner.

10. The swiveling table system of claim 1, further comprising a first 2D sensor configured to capture a 2D image of a front view of the fabric when the fabric is rotating.

11. The swiveling table system of claim 1, further comprising a second 2D sensor configured to capture a 2D image of a top view of the fabric when the fabric is placed on the coplanar.

12. The swiveling table system of claim 1, further comprising a backplane vertically disposed on the base, wherein the backplane and the base form an “L” shape appearance.

13. The swiveling table system of claim 12, wherein the backplane and the base are configure to be a green screen.

14. A method for operating the swiveling table system as claimed in claim 1 to build a 3D model of a fabric, comprising:
making the outer ring tray coplanar with the central tray;
placing the fabric on the coplanar;
controlling the lifting column to lower the outer ring tray in a free-fall manner to make the outer ring tray drop from a position coplanar with the central tray to the descending position in the direction perpendicular to the base;
activating the central rotating column to rotate the central tray to rotate the fabric;
activating the 3D sensor to capture the rotating image of the fabric; and
building the 3D model of the fabric according to the rotating image of the fabric.

15. The method of claim 14, wherein making the outer ring tray coplanar with the central tray further comprises making the outer ring tray surround the central tray and not coupled with the central tray.

16. The method of claim 15, wherein the central tray includes a fabric plate and a pressure plate, and placing the fabric on the coplanar comprises:
placing the fabric on the fabric plate; and
fixing the pressure plate on the fabric plate in a magnetically attractive manner to fix the fabric on the fabric plate.

17. The method of claim 16, further comprising forming warp scales on the fabric plate.

18. The method of claim 17, wherein placing the fabric on the fabric plate comprises positioning warp and weft directions of the fabric according to the warp scales.

19. The method of claim 14, further comprising activating a first 2D sensor to capture a 2D image of a front view of the fabric after activating the central rotating column to rotate the central tray to rotate the fabric.

20. The method of claim 14, further comprising activating a second 2D sensor to capture a 2D image of a top view of the fabric after placing the fabric on the coplanar.
